(19) United States
(12) Patent Application Publication
(10) Pub. No.: US 2025/0263547 A1
(43) Pub. Date: Aug. 21, 2025

(54) NON-DRYING ELECTROLYTE GELS FOR SOFT ELECTRODES

(71) Applicant: Meta Platforms Technologies, LLC, Menlo Park, CA (US)

(72) Inventors: Jitkanya WONG, Everett, WA (US); Thomas John Farrell WALLIN, Redmond, WA (US); Li YAO, Redmond, WA (US); Pinghung WEI, Kirkland, WA (US); Fan WANG, Kirkland, WA (US)

(21) Appl. No.: 18/151,693

(22) Filed: Jan. 9, 2023

Related U.S. Application Data
(60) Provisional application No. 63/266,813, filed on Jan. 14, 2022.

(51) Int. Cl. C08L 39/04; A61B 5/266; C08F 2/50; C08F 26/06

(52) U.S. Cl. CPC C08L 39/04; A61B 5/266; C08F 2/50; C08F 26/06; A61B 2562/0217; A61B 2562/125

(57) ABSTRACT

Described herein are non-drying, ionic liquid-based electrolyte gels (ion gel) fabricated using a mixture of free and polymerizable imidazolium bistriflimide (TFSI) ionic liquids and polyurethane diacrylate crosslinker. The ionic liquid-based electrolyte gels as described herein exhibit comparable softness with epidermal skin and water-based electrolyte gels, with suitable storage moduli values. The ion gels described herein not dry out when exposed to air and has stable storage modulus values, thus making it possible to be used long term and in a wide range of temperatures.

### CROSS-REFERENCE TO PRIORITY APPLICATION

This application claims the benefit of and priority to U.S. Provisional Patent Application No. 63/266,813, filed Jan. 14, 2022, the contents of which are herein incorporated by reference in its entirety.

### BACKGROUND

EMG electrodes convert motor neuron signals into electrical signals. The electrical signals are then processed and amplified for external device control. Previously used technologies have not been effective. Metal electrodes have been directly used for many applications; however, such electrodes are rigid and thus are not comfortable to be worn for prolonged periods (e.g., a full day). Wet electrolyte gels have been developed to mimic the protective and sensory functions of the human skin, resulting in reduced contact impedance and higher signal-to-noise ratio than metal electrodes. However, hydrogels have problems such as rapid drying and poor skin adhesion, resulting in narrow working temperature range and limitations of the materials for short-term use only.

### SUMMARY

Described herein are non-drying, ionic liquid-based electrolyte gels (ion gel) fabricated using a mixture of free and polymerizable imidazolium bistriflimide (TFSI) ionic liquids and polyurethane diacrylate crosslinker. The ionic liquid-based electrolyte gels as described herein exhibit comparable softness with epidermal skin and water-based electrolyte gels, with suitable storage moduli values as described below. Importantly and surprisingly, the ion gel does not dry out when exposed to air and has stable storage modulus values, thus making it possible to be used long term and in a wider range of temperatures.

The novel ion gel formulations are useful, for example, for developing soft electrodes, soft actuators, and soft sensors, among other uses. The formulations described herein maximize the performance of electrodes, actuators, sensors, and the like, as soft materials with high conductivity as well as good compatibility with human body/skin are needed. The formulations described herein deliver this unique combination of properties. The ionic conductivities for these gels are in the same range as commercial hydrogel electrodes, as further detailed herein. A final gel electrode has been fabricated and shown to have comparable/lower skin contact impedance values than commercial hydrogel electrodes.

Specifically, described herein is an ion gel resin formulation, comprising a polymerizable ionic liquid, a nonpolymerizable ionic liquid, or a combination thereof; and a crosslinker. Optionally, the polymerizable ionic liquid is present in an amount up to 95 wt. % based on the weight of the ion gel resin formulation. For example, the polymerizable ionic liquid can be present in an amount of from 20 wt. % to 95 wt. % based on the weight of the ion gel resin formulation. Optionally, the nonpolymerizable ionic liquid is present in an amount up to 95 wt. % based on the weight of the ion gel resin formulation. For example, the nonpolymerizable ionic liquid can be present in an amount of from 20 wt. % to 95 wt. % based on the weight of the ion gel resin formulation. In some examples, the polymerizable ionic liquid and the nonpolymerizable ionic liquid are present in the ion gel resin formulation. Optionally, the polymerizable ionic liquid comprises a bis(trifluoromethanesulfonyl)imide (TFSI) anion. Optionally, the nonpolymerizable ionic liquid comprises a bis(trifluoromethanesulfonyl)imide (TFSI) anion.

Optionally, the polymerizable ionic liquid comprises a compound of the following formula:

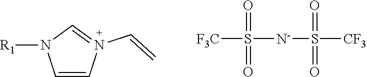

wherein R₁ is a C₁-C₈ alkyl or an epoxide monomer or repeating unit.

Optionally, the nonpolymerizable ionic liquid comprises a compound of the following formula:

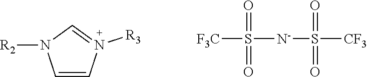

wherein R₂ and R₃ are each independently a C₁-C₈ alkyl or an epoxide monomer or repeating unit.

The crosslinker can optionally comprise a urethane polymer (e.g., a urethane acrylate polymer). The crosslinker can be present in an amount of from 1 wt. % to 15 wt. % based on the weight of the ion gel resin formulation. Optionally, the crosslinker is present in an amount of from 5 wt. % to 10 wt. % based on the weight of the ion gel resin formulation.

Optionally, the crosslinker comprises a compound of the following formula:

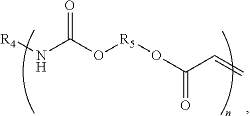

wherein:
R₄ and R₅ are each independently selected from substituted or unsubstituted alkyl, substituted or unsubstituted heteroalkyl, substituted or unsubstituted aryl, substituted or unsubstituted heteroaryl, and n is 2 or greater.

Optionally, the crosslinker comprises a compound of the following formula:

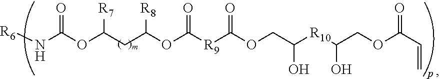

wherein:
R₆ is a substituted or unsubstituted alkyl; R₇ and R₈ are each independently selected from hydrogen or substituted or unsubstituted alkyl; m is 0, 1, 2, 3, 4, 5, 6, 7, 8, 9, 10, or greater; R₉ and R₁₀ are each independently selected from substituted or unsubstituted alkyl, substituted or unsubstituted alkenyl, substituted or unsubstituted cycloalkyl, substituted or unsubstituted cycloalkenyl, substituted or unsubstituted aryl, and substituted or unsubstituted heteroaryl; and p is 2 or greater.

The ion gel resin formulation can further comprise a photoinitiator. In some examples, the photoinitiator comprises a phosphine oxide photoinitiator (e.g., phenylbis(2,4,6-trimethylbenzoyl)phosphine oxide (BAPO)). The photoinitiator can be present in an amount of 0.05 wt. % to 0.15 wt. % based on the weight of the ion gel resin formulation. The ion gel resin formulation described herein can be free from water or other aqueous solvents.

Also described herein is a molded product comprising an ion gel resin formulation as described herein. Optionally, the molded product is an electrode. Further described herein is a wearable device, comprising a molded product as described herein integrated into the device.

Additionally described herein are methods of preparing a molded product, comprising mixing a polymerizable ionic liquid, a nonpolymerizable ionic liquid, or a combination thereof; and a crosslinker to form an ion gel resin formulation; and subjecting the ion gel resin formulation to an external stimulus. Optionally, the external stimulus is a light source emitting light at a wavelength of from 350 to 410 nm.

The details of one or more embodiments are set forth in the description below. Other features, objects, and advantages will be apparent from the description and from the claims.

### BRIEF DESCRIPTION OF THE DRAWINGS

FIG. 1 is a depiction of a polymer gel network based on the ionic liquids and polyurethane crosslinkers as described herein.

FIG. 2 is a graph depicting the porcine skin contact impedance results, comparing formulations as described herein with a commercially available sensor.

### DETAILED DESCRIPTION

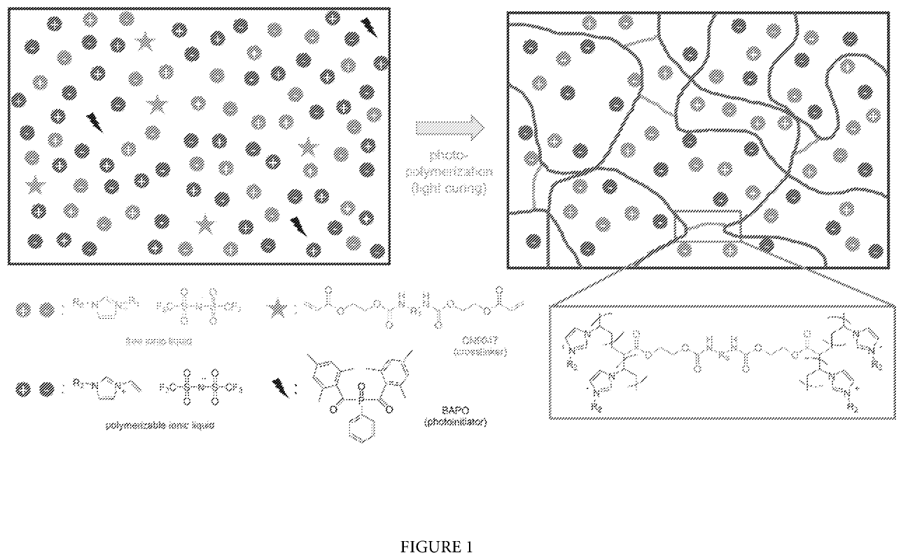

Described herein are novel ion gel formulations and methods of making the same. The ion gel formulations include polymerizable and/or nonpolymerizable ionic liquids and a crosslinker (e.g., a polyurethane crosslinker). The formulations are used to prepare elastomers which are soft, stretchable, hydrophobic, and exhibits good ionic conductivity. See FIG. 1. The ionic liquids' good solubility allows the components to be mixed without the need for a co-solvent. Due to its low apparent viscosity as measured by a rheometer, the formulation can be poured into a mold or stereolithography (SLA)-printed, for example, to form the shape of the cured material. In some cases, the apparent viscosity values are less than 50 Pa·s, less than 10 Pa·s, less than 5 Pa·s, or less than 1 Pa·S at 1% strain and 1 Hz. For example, the formulation can be SLA-printed to create complex topology to increase the contact surface and achieve low skin-contact electrical impedance for EMG electrode applications. Overall, the ion gels resulting from the formulations described herein can efficiently reduce the contact electrical impedance to improve EMG signals, provide comfort to user, and solve the issue of drying over long-term use.

The ion gel formulations described herein include hydrophobic ionic liquids and crosslinkers, such as polyurethane acrylate crosslinkers, and result in water-free soft gels. The hydrophobic ionic liquid can include a nonpolymerizable ionic liquid, a polymerizable ionic liquid, or a combination of these components. The nonpolymerizable, free ionic liquid contributes to the softness and ionic conductivity of the material, while the polymerizable ionic liquid contributes to mechanical robustness and retention of the free ionic liquid within the material matrix.

The ionic liquids described herein are known to have negligible vapor pressure, thus solving the issue of solvent evaporation in hydrogels. Having the same ionic conductivity mechanism as motor neuron signals, the ion gels to transmit EMG signal with minimal loss in resolution. Unlike most ion gels, the hydrophobicity of the bis(trifluoromethanesulfonyl)imide (also referred to herein as “bistriflimide” or “TFSI”) anion prevents water pickup/swelling of the material.

A polymerizable ionic liquid suitable for use in the formulations described herein includes, for example, an ionic liquid including a bis(trifluoromethanesulfonyl)imide (TFSI) anion. In some examples, a suitable polymerizable ionic liquid includes an imidazolium cation substituted with at least one polymerizable moiety, such as an alkenyl group. In some examples, the polymerizable ionic liquid is as depicted below in Formula I:

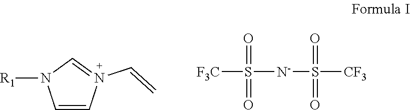

In Formula I, R₁ is a C₁-C₈ alkyl group or an epoxide monomer or repeating unit. For example, R₁ can be methyl (CH₃), ethyl (CH₃CH₂—), propyl (CH₃CH₂CH₂—), butyl (CH₃CH₂CH₂CH₂—), pentyl (CH₃(CH₂)₃CH₂—), hexyl (CH₃(CH₂)₄CH₂—), heptyl (CH₃(CH₂)₅CH₂—), or octyl (CH₃(CH₂)₆CH₂—). Optionally, R¹ can be an ethylene oxide repeating unit or propylene oxide repeating unit.

In some examples, a suitable polymerizable ionic liquid is 1-butyl-3-vinylimidazolium bis(trifluoromethanesulfonyl)imide ([BVIM]BF₄), which contains an imidazolium cation and bis(trifluoromethanesulfonyl)imide anion. The imidazolium in [BVIM]BF₄ has a vinyl group and a butyl group attached to one of the two nitrogens on the 5-membered ring, as depicted below:

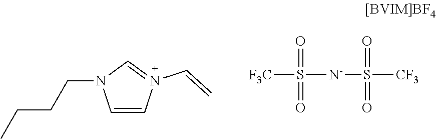

Optionally, the polymerizable ionic liquid is present in an amount up to 95 wt. % based on the weight of the ion gel resin formulation. For example, the polymerizable ionic liquid can be present in an amount of from 20 wt. % to 95 wt. % based on the weight of the ion gel resin formulation. In some cases, the polymerizable ionic liquid is present in an amount of from 25 wt. % to 90 wt. %, from 30 wt. % to 85 wt. %, from 35 wt. % to 80 wt. %, from 40 wt. % to 75 wt. %, or from 45 wt. % to 70 wt. % based on the weight of the ion gel resin formulation. For example, the polymerizable ionic liquid can be present in an amount of 5 wt. %, 10 wt. %, 20 wt. %, 25 wt. %, 30 wt. %, 35 wt. %, 40 wt. %, 45 wt. %, 50 wt. %, 55 wt. %, 60 wt. %, 65 wt. %, 70 wt. %, 75 wt. %, 80 wt. %, 85 wt. %, 90 wt. %, or 95 wt. %, based on the weight of the ion gel resin formulation.

A nonpolymerizable ionic liquid suitable for use in the formulations described herein includes, for example, an ionic liquid including a bis(trifluoromethanesulfonyl)imide (TFSI) anion. In some examples, a suitable nonpolymerizable ionic liquid includes an imidazolium cation that is optionally substituted with a group. However, the substituents of the imidazolium cation (or other cation for use in the ionic liquid) are not polymerizable. In some examples, the nonpolymerizable ionic liquid is as depicted below in Formula II:

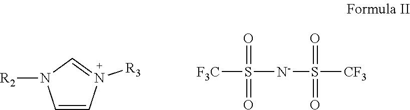

In Formula II, R₂ and R₃ are each independently a C₁-C₈ alkyl group or an epoxide monomer or repeating unit. For example, R₂ or R₃ can be methyl (CH₃), ethyl (CH₃CH₂—), propyl (CH₃CH₂CH₂—), butyl (CH₃CH₂CH₂CH₂—), pentyl (CH₃(CH₂)₃CH₂—), hexyl (CH₃(CH₂)₄CH₂—), heptyl (CH₃(CH₂)₅CH₂—), or octyl (CH₃(CH₂)₆CH₂—). Optionally, R₂ and/or R₃ can be an ethylene oxide repeating unit or propylene oxide repeating unit.

In some examples, a suitable nonpolymerizable ionic liquid is 1-butyl-3-methylimidazolium bis(trifluoromethanesulfonyl)imide ([BMIM]BF₄), which contains an imidazolium cation and bis(trifluoromethanesulfonyl)imide anion. The imidazolium in [BMIM]BF₄ has a butyl group and a methyl group attached to one of the two nitrogens on the 5-membered ring, as depicted below:

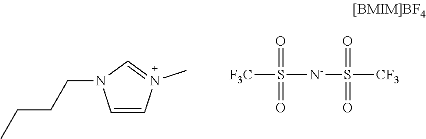

Optionally, the nonpolymerizable ionic liquid is present in an amount up to 95 wt. % based on the weight of the ion gel resin formulation. For example, the nonpolymerizable ionic liquid can be present in an amount of from 20 wt. % to 95 wt. % based on the weight of the ion gel resin formulation. In some cases, the nonpolymerizable ionic liquid is present in an amount of from 25 wt. % to 90 wt. %, from 30 wt. % to 85 wt. %, from 35 wt. % to 80 wt. %, from 40 wt. % to 75 wt. %, or from 45 wt. % to 70 wt. % based on the weight of the ion gel resin formulation. For example, the nonpolymerizable ionic liquid can be present in an amount of 5 wt. %, 10 wt. %, 20 wt. %, 25 wt. %, 30 wt. %, 35 wt. %, 40 wt. %, 45 wt. %, 50 wt. %, 55 wt. %, 60 wt. %, 65 wt. %, 70 wt. %, 75 wt. %, 80 wt. %, 85 wt. %, 90 wt. %, or 95 wt. %, based on the weight of the ion gel resin formulation.

In some examples, the polymerizable ionic liquid and the nonpolymerizable ionic liquid are present in the ion gel resin formulation.

The ion gel resin formulations described herein also include a crosslinker. The crosslinker further enhances the shape integrity of the material, and, surprisingly, at the same time reduces the overall stiffness.

In some cases, the crosslinker is a polyurethane crosslinker, such as a polyurethane diacrylate crosslinker. The polyurethane crosslinkers contain urethane segments, which can provide shape memory sites while at the same time impart softness and stretchability to the material.

Optionally, the crosslinker for use in the ion gel resin formulations described herein can be a urethane acrylate oligomer that can include at least one urethane linkage in the backbone and at least two acrylate functional groups in the chain end. In some examples, a crosslinker for use in the ion gel resin formulation can include a compound of Formula III:

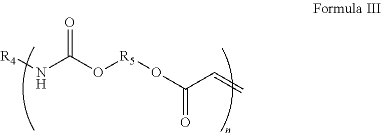

In Formula III, R₄ and R₅ are each independently selected from substituted or unsubstituted alkyl, substituted or unsubstituted heteroalkyl, substituted or unsubstituted aryl, substituted or unsubstituted heteroaryl. For example, R₄ and/or R₅ can be any (hetero) hydrocarbyl group, including aliphatic and aromatic groups.

Also in Formula III, n is 2 or greater (e.g., 2 to 20, 3 to 18, 4 to 16, or 5 to 15). For example, n can be 2, 3, 4, 5, 6, 7, 8, 9, 10, 11, 12, 13, 14, 15, or greater than 15.

In other cases, the crosslinker for use in the ion gel resin formulations described herein can be a urethane acrylate oligomer according to Formula IV:

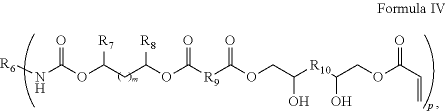

In Formula IV, R₆ is a substituted or unsubstituted alkyl. In Formula III, R⁶ is attached to (i.e., carrying) “n” number of —NH— groups.

Also in Formula IV, R₇ and R₈ are each independently selected from hydrogen or substituted or unsubstituted alkyl (e.g., alkyl or hydroxyalkyl). For example, in Formula III, R₇ and R₇ are each independently a C₁-C₈ alkyl group (e.g., a C₁-C₃ alkyl group). For example, R₇ or R₈ can be methyl (CH₃), ethyl (CH₃CH₂—), propyl (CH₃CH₂CH₂—), butyl (CH₃CH₂CH₂CH₂—), pentyl (CH₃(CH₂)₃CH₂—), hexyl (CH₃(CH₂)₄CH₂—), heptyl (CH₃(CH₂)₅CH₂—), or octyl (CH₃(CH₂)₆CH₂—). R₇ and R₈ can be identical moieties or can be different moieties.

Additionally in Formula IV, m is 0, 1, 2, 3, 4, 5, 6, 7, 8, 9, 10, or greater.

Further in Formula IV, R₉ can be substituted or unsubstituted alkyl, substituted or unsubstituted alkenyl, substituted or unsubstituted cycloalkyl, substituted or unsubstituted cycloalkenyl, substituted or unsubstituted aryl, or substituted or unsubstituted heteroaryl. Optionally, R₉ can be an alkylene radical, a cycloalkylene radical, or an arylene radical. In some examples, R₉ can be substituted by CH₃, Et, CH₃—(CH₂) n (where n>1), H, OH, OMe, OEt, OiPr, F, Cl, Br, I, Ph, NO₂, SO₃, SO₂Me, iPr, t-Bu, sec-Bu, Et, acetyl, SH, SMe, carboxyl, aldehyde, amide, nitrile, ester, SO₂NH₃, NH₂, NMe₂, NMeH, or C₂H₂. The substitution can occur at any position where the molecule could be substituted by one of the above functional groups as determined by one of ordinary skill in the art based on the guidance in the present application.

Also in Formula IV, R₁₀ can be substituted or unsubstituted alkyl, substituted or unsubstituted alkenyl, substituted or unsubstituted cycloalkyl, substituted or unsubstituted cycloalkenyl, substituted or unsubstituted aryl, or substituted or unsubstituted heteroaryl. Optionally, R₁₀ can be an alkylene radical, a cycloalkylene radical, or an arylene radical. In some examples, R₁₀ can be substituted by CH₃, Et, CH₃—(CH₂)ₙ (where n>1), H, OH, OMe, OEt, OiPr, F, Cl, Br, I, Ph, NO₂, SO₃, SO₂Me, iPr, t-Bu, sec-Bu, Et, acetyl, SH, SMe, carboxyl, aldehyde, amide, nitrile, ester, SO₂NH₃, NH₂, NMe₂, NMeH, or C₂H₂. The substitution can occur at any position where the molecule could be substituted by one of the above functional groups as determined by one of ordinary skill in the art based on the guidance in the present application.

Additionally in Formula IV, p can be 2 or greater (e.g., 2 to 20, 3 to 18, 4 to 16, or 5 to 15). For example, p can be 2, 3, 4, 5, 6, 7, 8, 9, 10, 11, 12, 13, 14, 15, or greater than 15.

Optionally, the crosslinker can be obtained from commercial sources. In some examples, the crosslinker can be CN9047, a low viscosity aliphatic urethane acrylate oligomer commercially available from Sartomer (Exton, PA).

Optionally, the crosslinker can be present in an amount of from 1 wt. % to 15 wt. % based on the weight of the ion gel resin formulation. In some cases, the crosslinker is present in an amount of from 2 wt. % to 14 wt. %, from 3 wt. %, to 13 wt. %, from 4 wt. % to 12 wt. %, or from 5 wt. % to 10 wt. % based on the weight of the ion gel resin formulation. For example, the crosslinker can be present in an amount of 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. %, based on the weight of the ion gel resin formulation.

The ion gel resin formulation can further comprise a photoinitiator. The photoinitiator chemically initiates the polymerization of a photoactive polymerizable material. The photoinitiator generally should offer a source of species that initiate polymerization of the particular photoactive polymerizable material, e.g., photoactive monomer. In some examples, the photoinitiators used herein are sensitive to ultraviolet and visible radiation of from about 200 nm to about 800 nm (e.g., from about 250 nm to about 700 nm, from about 300 nm to about 600 nm, or from about 350 nm to about 550 nm).

A variety of photoinitiators known to those skilled in the art and available commercially are suitable for use as described herein, for example, those including a phosphine oxide group. In some examples, the photoinitiator comprises a phosphine oxide photoinitiator (e.g., phenylbis(2,4,6-trimethylbenzoyl)phosphine oxide (BAPO)). BAPO is a radical polymerization initiator. When added to the formulation and exposed to 365-405 nm light, BAPO can absorb photons and produce active radicals to initiate radical polymerization of the polymerizable ionic liquid and urethane diacrylate crosslinker.

Optionally, the photoinitiator can be obtained from commercial sources. In some examples, the photoinitiator can be BAPO commercially available from Millipore Sigma (St. Louis, MO).

The photoinitiator can be present in an amount of 0.05 wt. % to 0.15 wt. % based on the weight of the ion gel resin formulation. For example, the photoinitiator can be present in an amount of 0.05 wt. %, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, or 0.15 wt. % based on the weight of the ion gel resin formulation.

The ion gel resin formulation described herein can be free from water or other aqueous solvents.

Optionally, the ion gel resin formulations described herein can additionally include one or more additives. In some examples, the formulations described herein plasticizers for altering the properties of a molded product according to the present disclosure, including the melting point, flexibility, toughness, diffusibility of the monomers and/or oligomers, and ease of processability. Examples of suitable plasticizers include dibutyl phthalate, poly (ethylene oxide) methyl ether, N,N-dimethylformamide, etc. Plasticizers differ from solvents in that solvents are typically evaporated whereas plasticizers are meant to remain in the molded product.

Other types of additives for optional use in the ion gel resin formulations and molded products described herein include inert diffusing agents having relatively high or low refractive indices. Other additives that may be used in the ion gel resin formulations and molded products of the present disclosure include: pigments, fillers, nonphotoinitiating dyes, antioxidants, bleaching agents, mold releasing agents, antifoaming agents, infrared/microwave absorbers, surfactants, adhesion promoters, etc.

Suitable polymerization retarders and inhibitors for use herein include but are not limited to one or more of the following: for free radical polymerizations, various phenols including butylated hydroxytoluenes (BHT) such as 2,6-di-t-butyl-p-cresol, p-methoxyphenol, diphenyl-p-benzoquinone, benzoquinone, hydroquinone, pyrogallol, resorcinol, phenanthraquinone, 2,5-toluquinone, benzylaminophenol, p-dihydroxybenzene, 2,4,6-trimethylphenol, etc.; various nitrobenzenes including o-dinitrobenzene, p-dinitrobenzene, m-dinitrobenzene, etc.; N-phenyl-1-naphthylamine, N-phenyl-2-naphthylamine, cupferron, phenothiazine, tannic acid, p-nitrosamine, chloranil, aniline, hindered anilines, ferric chloride, cupric chloride, triethylamine, etc. These polymerization retarders and inhibitors can be used individually (e.g., a single retarder) or in combinations of two or more, e.g., a plurality of retarders.

In some cases, polymerization reactions involving both polymerization retarders and inhibitors should lead to termination reactions. If reinitiation occurs to any appreciable degree, then the agent is typically considered a chain transfer agent. For example, triethylamine can be used as a chain transfer agent since it is also capable of reinitiating some radical polymerizations; however, when the reinitiation is slow compared to termination reactions, then even chain transfer agents can be considered potential polymerization retarders or inhibitors for the purposes of the present disclosure. Suitable chain transfer agents for use herein include but are not limited to: triethylamine, thioethers, compounds having carbonate groups, ethers, toluene derivatives, allyl ethers, etc. Chain transfer agents that are mildly retarding can be desirable because these can be incorporated into the matrix and enable attachment of the polymers and photoinitiator radicals to the matrix.

In addition to retarders, inhibitors and/or chain transfer agents, metastable reactive centers and light labile phototerminators can also be used to control polymerization reactions described herein of the appropriate reactivity. For example, nitroxyl radicals can be added as a metastable reactive center. Nitroxyl radicals create pseudo-living radical polymerizations with certain monomers. Thus, the nitroxyl radical initially behaves as a terminating agent (such as an inhibitor); however, depending on the temperature at which the polymerization is carried out, the termination is reversible. Other potential metastable reactive center include triphenylmethyl radicals, dithioesters are typically used in Reversible Addition-Fragmentation chain Transfer (RAFT) polymerizations, that can behave as appropriate metastable reactive centers, etc.

Use of a light labile phototerminator provides the ability to control the activity of the reactive species with light. A light labile phototerminator is any molecule capable of undergoing reversible termination reactions using a light source. For example, certain cobaltoxime complexes can be used to photoinitiate radical polymerizations, and yet, also terminate the same radical polymerizations.

Dithioesters are also suitable as light labile phototerminators because they can reversibly form radicals with appropriate wavelengths of light. Under the appropriate conditions and with appropriate monomers (such as styrenes and acrylates), it is possible to restart the polymerization by irradiating with a photoinitiating light source (e.g., recording light). Thus, as long as a given volume is exposed to a photoinitiating light source, radical polymerization continues, whereas when the photoinitiating light is off or absent, the polymerizations are terminated.

Certain monomer mixtures can also behave in a manner that can control the degree or rate of polymerization. For example, if a small amount of alpha methyl styrene is present in an acrylate polymerization, the acrylate will add into the alpha methyl styrene and the styrene will not substantially reinitiate polymerization of the acrylate, e.g., the alpha methyl styrene retards the rate of acrylate polymerization. Additionally, the alpha methyl styrene is slow to polymerize with itself, and thus behaves as a polymerization retarder/inhibitor even though it is a comonomer.

Additionally described herein are methods of preparing a molded product, comprising mixing a polymerizable ionic liquid, a nonpolymerizable ionic liquid, or a combination thereof and a crosslinker to form an ion gel resin formulation; and subjecting the ion gel resin formulation to an external stimulus.

The mixing can be performed using any suitable apparatus for the selected components, along with the selected amounts (e.g., laboratory-scale or process-scale). In some examples, the mixing can be performed using speed-mixing, internal mixing, ball milling, planetary milling, roll-milling, or an attritor. The mixing is performed for a suitable period of time to combine the materials into the ion gel resin formulation.

Following the mixing, the ion gel resin formulation is exposed to an external stimulus. Optionally, the external stimulus is a light source emitting light at a wavelength of from 200 nm to 800 nm (e.g., 250 nm to 700 nm, 300 nm to 650 nm, or 350 to 550 nm).

As described above, with the addition of a biocompatible photoinitiator (e.g., bis-acylphosphine oxide (BAPO)), the material can be cured with light exposure (e.g., light exposure of at least 350 nm). Free radical polymerization allows the polymerizable ionic liquids and crosslinker to form a charged network with maximized intermolecular interactions to the free ionic liquid. Via ion-dipole and hydrophobic interactions, the polymerized ionic liquid network prevents the free ionic liquids from leaching out onto the skin via contact.

Prior to light exposure, the formulations described herein can be poured into a mold to achieve the desired shape. For example, the formulations can be poured into a silicone rubber mold and exposed to light of the appropriate wavelength (as described above) for a period of time to cure the material. In some examples, the period of time for curing can be 20 minutes or more, 30 minutes or more, 40 minutes or more, 50 minutes or more, 60 minutes or more, or 70 minutes or more. Optionally, the curing time is from 10 minutes to 70 minutes, from 20 minutes to 60 minutes, or from 30 minutes to 50 minutes. The formulations described herein can also be used as a resin for lithography (e.g., stereolithography or SLA) printing.

The molded product can have any suitable shape, and can be dictated by the end use of the product. In some cases, the molded product can have a cylindrical shape. Optionally, the cylindrical shape can have a diameter ranging from, for example, 1 mm to 20 mm (3 mm to 15 mm). Other suitable shapes include, for example, a flat shape, a microneedle, a fuzzy structure, a dome shape, a hollow structure, or a cone shape. Optionally, the molded product can be an electrode.

Optionally, the processing can further comprise coating one or more surfaces of the material with a polymer or another coating material. In some cases, at least one surface of the molded product is surface coated with a coating material. The coating material can be, for example poly (3,4-ethylenedioxythiophene) polystyrene sulfonate (PEDOT:PSS), a sulfonated tetrafluoroethylene based fluoropolymer-copolymer, a polymer binder, polyaniline, polypyrrole, silver nanowires (AgNW), gold nanowires (AuNW), liquid metal, or gold. In some cases, the coating can be performed by using dip-coating, ink-jet printing, slot-die coating, screen-printing, aerosol jetting, electrochemical deposition, or a surface treatment using oxygen plasma, a silane treatment, a corona surface treatment, or any other suitable method.

Optionally, the molded product exhibits an average storage modulus of 0.4 MPa or lower. For example, the average storage modulus of the molded product can be 0.005 MPa to 0.4 MPa, 0.01 MPa to 0.3 MPa, or 0.05 MPa to 0.2 MPa. The molded product can be stable up to temperatures of 110° C. For example, the molded product can be stable at temperatures of −10° C. to 110° C., from 0° C. to 100° C., from 10° C. to 90° C., from 20° C. to 80° C., or from 30° C. to 70° C. The ionic conductivity of the molded products described herein can range from 1×10⁻⁶ to 1×10⁻³ S/cm, at temperatures of 25° C. or 37° C. Suitable methods for determining the properties of the molded product are provided below in the Examples of the present application.

The molded products described herein can be integrated into a wearable device. Optionally, the wearable devices can be used to collect biopotential signals and/or electromyography signals. Suitable wearable devices include, for example, a wristband.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1: Ion Gel Formulation

Ion gel resins were formulated using the materials listed in Table 1. All components were combined and a speed mixer (SPEEDMIXER DAC 150.1 FVZ-K, FlackTek, Inc., Landrum, SC, ETS) was used to thoroughly mix the resins. The mixing was set at 1550 rpm/min for 35 sec, 2000 rpm/min for 20 seconds, and then 2000 rpm/min for 35 seconds without vacuum.

TABLE 1
Materials

| | Component Description |
|---|---|
| Urethane Acrylate 1 | Low viscosity aliphatic urethane acrylate oligomer |
| Photoinitiator | Phenylbis(2,4,6-trimethylbenzoyl)phosphine oxide (BAPO) |
| Polymerizable ionic | 1-butyl-3-vinylimidazolium bis(trifluoromethanesulfonyl)imide |
| liquid | ([BVIM]BF₄) |
| Nonpolymerizable | 1-butyl-3-methylimidazolium bis(trifluoromethanesulfonyl)imide |
| ionic liquid | ([BMIM]BF₄) |

The resins were poured into a silicone rubber mold and exposed to 365 nm light for 50 minutes to cure the material. The compositions of the prepared resins are shown below in Table 2.

TABLE 2
Compositions of Ion Gel Resins

| | Urethane | [BVIM]BF₄ | [BMIM]BF₄ | BAPO |
|---|---|---|---|---|
| | Acrylate (wt %) | (wt %) | (wt %) | (wt %) |
| Resin A1 | 5% | 94.9% | 0% | 0.1% |
| Resin A2 | 5% | 71.175% | 23.725% | 0.1% |
| Resin A3 | 5% | 47.45% | 47.45% | 0.1% |
| Resin A4 | 5% | 23.725% | 71.175% | 0.1% |
| Resin A5 | 5% | 0% | 94.9% | 0.1% |
| Resin B1 | 7.5% | 92.4% | 0% | 0.1% |
| Resin B2 | 7.5% | 69.3% | 23.1% | 0.1% |
| Resin B3 | 7.5% | 46.2% | 46.2% | 0.1% |
| Resin B4 | 7.5% | 23.1% | 69.3% | 0.1% |
| Resin B5 | 7.5% | 0% | 92.4% | 0.1% |
| Resin C1 | 10% | 89.9% | 0% | 0.1% |
| Resin C2 | 10% | 67.425% | 22.475% | 0.1% |
| Resin C3 | 10% | 44.95% | 44.95% | 0.1% |
| Resin C4 | 10% | 22.475% | 67.425% | 0.1% |
| Resin C5 | 10% | 0% | 89.9% | 0.1% |

### Example 2: Storage Modulus

The curing kinetics were measured using a parallel-plate geometry at 1% strain on an ARES Rheometer (TA Instruments, Wood Dale, IL, US) equipped with a forced convection oven accessory at oscillating mold at frequency of 1 Hz at 90° C. through a time sweep.

The samples from Table 2 were made by pouring resin into cylinder-shaped silicone rubber mold. The samples were 12 mm in diameter. The samples were then cured using 365 nm light for 50 minutes, and then placed on the compression clamp in the TA Instruments Dynamic Mechanical Analyzer (DMA 850) with preload of 0.01 N. During the test, the samples were heated from −10° C. to 110° C., cooled to −10° C., and heated to 110° C. The storage modulus was recorded at 1% oscillatory strain.

Table 3 shows the average storage modulus (oscillatory, compression) for the selected formulations, as well as the stable temperature range where the storage modulus is +/−10% MPa of the reported value.

TABLE 3
Mechanical Performance

| | | | Average | |
|---|---|---|---|---|
| | | | Storage | Stable |
| | % Polymerized | | Modulus | Temperature |
| | IL | % Crosslinker | (MPa) | Range (° C.) |
| A1 | 94.9 | 5 | 0.16333 | 67.88-88.43 |
| A3 | 47.45 | 5 | 0.06775 | −9.78-102.10 |
| A5 | 0 | 5 | 0.00569 | −9.64-108.44 |
| C1 | 89.9 | 10 | 0.32971 | 60.49-87.13 |
| C3 | 44.95 | 10 | 0.04868 | −9.62-68.41 |
| C5 | 0 | 10 | 0.02501 | −9.78-55.8 |

### Example 3: Ionic Conductivity

Ion gel samples from Table 2 were prepared by pouring the resin into a disc-shaped silicone rubber mold. The samples were 20 mm in diameter and 1-1.2 mm in thickness. Measurements were done on the TA Instruments ARES Rheometer with a Dielectric Thermal Analysis Accessory (DETA) parallel plate set up. The DETA was connected to LCR meter, and the conductance was measured over the range of 100-106 Hz applied potential at 25° C. and 37° C. The ionic conductivity was calculated by multiplying the conductance at a specific frequency by the sample's thickness and divided by the sample's cross-sectional area.

Table 4 shows the ionic conductivity values for the selected formulations at room temperature (25° C.) and biological temperature (37° C.) at 100 Hz applied potential.

TABLE 4
Electrical Performance

| | | | Ionic | Ionic |
|---|---|---|---|---|
| | | | Conductivity | Conductivity |
| | % Polymerized | | at 25° C. | at 37° C. |
| | IL | % Crosslinker | (S/cm) | (S/cm) |
| A1 | 94.9 | 5 | 1.94 × 10⁻⁵ | 3.62 × 10⁻⁵ |
| A3 | 47.45 | 5 | 4.71 × 10⁻⁵ | 6.06 × 10⁻⁵ |
| A5 | 0 | 5 | 1.16 × 10⁻⁴ | 1.34 × 10⁻⁴ |
| C1 | 89.9 | 10 | 5.28 × 10⁻⁶ | 1.15 × 10⁻⁵ |
| C3 | 44.95 | 10 | 9.24 × 10⁻⁵ | 1.18 × 10⁻⁴ |
| C5 | 0 | 10 | 1.21 × 10⁻⁴ | 1.39 × 10⁻⁴ |

### Example 4: Porcine Skin Contact Impedance

Laboratory-grade porcine skin was thawed and soaked in PBS solution for 1 hour. Ion gel samples were then placed on the skin. An Ag/AgCl electrode was then placed on top of the ion gel and connected to the PalmSens4 Impedance Analyzer. 100 Hz potential was applied (EMG signal frequency), and the skin contact impedance was recorded over 5 minutes in ambient conditions.

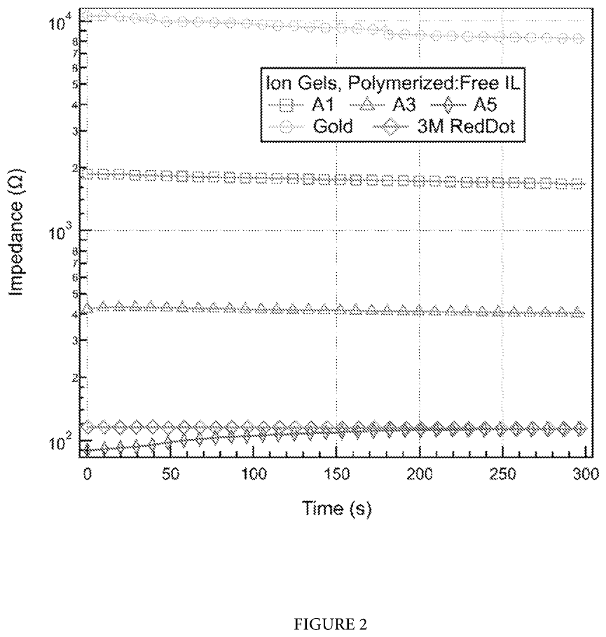

The results from the porcine skin contact impedance testing are shown in FIG. 2. Specifically, FIG. 2 shows the porcine skin contact impedance recorded over time, compared to commercially available 3M RedDot EMG sensor. The geometries of the ion gels were the same as the 3M REDDOT (commercially available from 3M; St. Minnesota).

### Example 5: Porcine Skin Contact Gel Fraction

Laboratory-grade porcine skin is thawed and soaked in PBS solution for 1 hour. The skin is then kept in an environmental chamber at 35% RH, 37° C. The ion gels (10 mm diam., 5 mm thick) are then placed on the porcine skin with weight on top, and the mass is monitored over time. Table 5 shows the gel fraction results via porcine skin contact for the selected formulations at room temperature (25° C.) and biological temperature (37° C.) at 100 Hz applied potential.

TABLE 5
Gel Fraction via Porcine Skin Contact

| | Day 0 | Day 1 | Day 4 | Day 9 |
|---|---|---|---|---|
| A1 | 100 | 100.0467 | 99.67759 | 99.37681 |
| A3 | 100 | 97.02417 | 99.4612 | 98.96805 |
| A5 | 100 | 103.7188 | 97.43461 | 98.31799 |
| C1 | 100 | 100.5567 | 99.86226 | 99.43486 |
| C3 | 100 | 99.93597 | 99.64429 | 99.22863 |
| C5 | 100 | 101.0086 | 99.02827 | 98.90494 |
| 3M REDDOT | 100 | 83.01785 | 100.8168 | 190.7325 |

Table 6 shows the gel fraction results via submersion in PBS (control). The A5 sample was mechanically weak and was destroyed after the submersion process.

TABLE 6
Gel Fraction via Submersion in PBS

| | Day 0 | Day 1 | Day 4 | Day 9 |
|---|---|---|---|---|
| A1 | 100 | 94.83375 | 93.65439 | 96.41207 |
| A3 | 100 | 91.18203 | 84.26709 | 81.61501 |
| C1 | 100 | 94.02962 | 92.31717 | 92.65521 |
| C3 | 100 | 91.08496 | 83.58184 | 83.0298 |
| C5 | 100 | 89.7675 | 79.45914 | 74.70908 |

The compositions and methods of the appended claims are not limited in scope by the specific compositions and methods described herein, which are intended as illustrations of a few aspects of the claims and any compositions and methods that are functionally equivalent are within the scope of this disclosure. Various modifications of the compositions and methods in addition to those shown and described herein are intended to fall within the scope of the appended claims. Further, while only certain representative compositions, methods, and aspects of these compositions and methods are specifically described, other compositions and methods are intended to fall within the scope of the appended claims. Thus, a combination of steps, elements, components, or constituents can be explicitly mentioned herein; however, all other combinations of steps, elements, components, and constituents are included, even though not explicitly stated.

What is claimed is:

1. An ion gel resin formulation, comprising:
a polymerizable ionic liquid, a nonpolymerizable ionic liquid, or a combination thereof; and
a crosslinker.

2. The ion gel resin formulation of claim 1, wherein the polymerizable ionic liquid is present in an amount up to 95 wt. % based on the weight of the ion gel resin formulation.

3. The ion gel resin formulation of claim 2, wherein the polymerizable ionic liquid is present in an amount of from 20 wt. % to 95 wt. % based on the weight of the ion gel resin formulation.

4. The ion gel resin formulation of claim 1, wherein the nonpolymerizable ionic liquid is present in an amount up to 95 wt. % based on the weight of the ion gel resin formulation.

5. The ion gel resin formulation of claim 4, wherein the nonpolymerizable ionic liquid is present in an amount of from 20 wt. % to 95 wt. % based on the weight of the ion gel resin formulation.

6. The ion gel resin formulation of claim 1, wherein the polymerizable ionic liquid and the nonpolymerizable ionic liquid are present in the ion gel resin formulation.

7. The ion gel resin formulation of claim 1, wherein the polymerizable ionic liquid comprises a bis(trifluoromethanesulfonyl)imide (TFSI) anion.

8. The ion gel resin formulation of claim 7, wherein the polymerizable ionic liquid comprises a compound of the following formula: 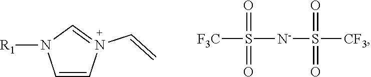
wherein
R₁ is a C₁-C₈ alkyl or an epoxide monomer or repeating unit.

9. The ion gel resin formulation of claim 1, wherein the nonpolymerizable ionic liquid comprises a bis(trifluoromethanesulfonyl)imide (TFSI) anion.

10. The ion gel resin formulation of claim 9, wherein the nonpolymerizable ionic liquid comprises a compound of the following formula: 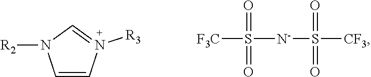
wherein
R₂ and R₃ are each independently a C₁-C₈ alkyl or an epoxide monomer or repeating unit.

11. The ion gel resin formulation of claim 1, wherein the crosslinker comprises a urethane polymer.

12. The ion gel resin formulation of claim 11, wherein the urethane polymer is a urethane acrylate.

13. The ion gel resin formulation of claim 1, wherein the crosslinker is present in an amount of from 1 wt. % to 15 wt. % based on the weight of the ion gel resin formulation.

14. The ion gel resin formulation of claim 13, wherein the crosslinker is present in an amount of from 5 wt. % to 10 wt. % based on the weight of the ion gel resin formulation.

15. The ion gel resin formulation of claim 1, wherein the crosslinker comprises a compound of the following formula: 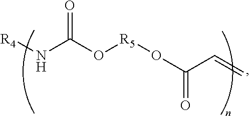
wherein:
R₄ and R₅ are each independently selected from substituted or unsubstituted alkyl, substituted or unsubstituted heteroalkyl, substituted or unsubstituted aryl, substituted or unsubstituted heteroaryl; and
n is 2 or greater.

16. The ion gel resin formulation of claim 1, wherein the crosslinker comprises a compound of the following formula: 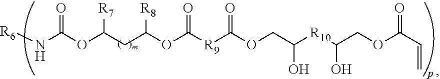
wherein:
R₆ is a substituted or unsubstituted alkyl;
R₇ and R₈ are each independently selected from hydrogen or substituted or unsubstituted alkyl;
m is 0, 1, 2, 3, 4, 5, 6, 7, 8, 9, 10, or greater;
R₉ and R₁₀ are each independently selected from substituted or unsubstituted alkyl, substituted or unsubstituted alkenyl, substituted or unsubstituted cycloalkyl, substituted or unsubstituted cycloalkenyl, substituted or unsubstituted aryl, and substituted or unsubstituted heteroaryl; and
p is 2 or greater.

17. The ion gel resin formulation of claim 1, further comprising a photoinitiator.

18. The ion gel resin formulation of claim 17, wherein the photoinitiator comprises a phosphine oxide photoinitiator.

19. The ion gel resin formulation of claim 18, wherein the phosphine oxide photoinitiator is phenylbis(2,4,6-trimethylbenzoyl)phosphine oxide (BAPO).

20. The ion gel resin formulation of claim 17, wherein the photoinitiator is present in an amount of 0.05 wt. % to 0.15 wt. % based on the weight of the ion gel resin formulation.

21. The ion gel resin formulation of claim 1, wherein the ion gel resin formulation is free from water or aqueous solvents.

22. A molded product, comprising an ion gel resin formulation according to claim 1.

23. The molded product of claim 22, wherein the molded product is an electrode.

24. A wearable device, comprising a molded product of claim 22 integrated into the wearable device.

25. A method of preparing a molded product of claim 22, comprising:
mixing a polymerizable ionic liquid, a nonpolymerizable ionic liquid, or a combination thereof; and a crosslinker to form an ion gel resin formulation; and
subjecting the ion gel resin formulation to an external stimulus.

26. The method of claim 25, wherein the external stimulus is a light source emitting light at a wavelength of from 350 to 410 nm.
